(19) United States
(12) Patent Application Publication
(10) Pub. No.: US 2016/0196649 A1
(43) Pub. Date: Jul. 7, 2016

(54) METHOD FOR DETERMINING AN INTRACORTICAL WORKING STATE OF A FUNCTIONAL NETWORK IN THE BRAIN

(71) Applicant: Siemens Healthcare GmbH, Erlangen (DE)

(72) Inventor: Tian Yi Qian, Beijing (CN)

(73) Assignee: Siemens Healthcare GmbH, Erlangen (DE)

(21) Appl. No.: 14/989,178

(22) Filed: Jan. 6, 2016

(30) Foreign Application Priority Data
Jan. 6, 2015 (CN) 201510003767.4

(51) Int. Cl. G06T 7/00; A61B 5/145; A61B 5/00; A61B 5/055

(52) U.S. Cl. CPC G06T 7/0012; A61B 5/055; A61B 5/14542; A61B 5/7271; A61B 5/4064; G06T 7/0081; G06T 2207/10088; G06T 2207/30016

(57) ABSTRACT

A method for determining an intracortical working state of a functional network of the brain includes acquiring a number of first blood oxygen saturation level time point vectors of a number of gray matter voxels of a cortex of a brain functional network template, each of the blood oxygen saturation level time point vectors respectively having blood oxygen saturation level signals of each of the gray matter voxels at a number of continuous time points in a particular time period. The method further includes clustering the blood oxygen saturation level time point vectors as a number of gray matter voxel cooperative time point categories by taking the blood oxygen saturation level signals as objects, the gray matter voxel cooperative time point categories being sets of a number of discrete time points of the gray matter voxels respectively in the particular time period. The method includes determining the number of gray matter voxel cooperative time point categories as the intracortical working state of the first cortex.

### BACKGROUND OF THE INVENTION

1. Field of the Invention

The present invention relates to the technical field of magnetic resonance imaging, and in particular to a method for determining an intracortical working state of a functional network of the brain using a magnetic resonance imaging system.

2. Description of the Prior Art

Magnetic resonance imaging (MRI) is a technology in which the phenomenon of magnetic resonance is utilized for the purpose of imaging. The basic principles of magnetic resonance are as follows: when an atomic nucleus contains a single proton, as is the case with the nuclei of the hydrogen atoms that are present throughout the human body, this proton exhibits spin motion and resembles a small magnet. The spin axes of these small magnets lack an adhesive pattern, but when an external magnetic field is applied, the small magnets will be rearranged according to the magnetic force lines of the external magnetic field. Specifically, they will align in two directions, either parallel or anti-parallel to the magnetic force lines of the external magnetic field. The direction parallel to the magnetic force lines of the external magnetic field is called the positive longitudinal axis, while the direction anti-parallel to the magnetic force lines of the external magnetic field is called the negative longitudinal axis. The atomic nuclei only have a longitudinal magnetization component, which has both a direction and a magnitude. A radio frequency (RF) pulse of a specific frequency is used to excite the atomic nuclei in the external magnetic field such that their spin axes deviate from the positive longitudinal axis or negative longitudinal axis, giving rise to resonance—this is the phenomenon of magnetic resonance. Once the spin axes of the excited atomic nuclei have deviated from the positive or negative longitudinal axis the atomic nuclei have a transverse magnetization component.

Once emission of the RF pulse has ended, the excited atomic nuclei emit an echo signal, gradually releasing the absorbed energy in the form of electromagnetic waves, such that their phase and energy level both return to the pre-excitation state. An image can be reconstructed by subjecting the echo signal emitted by atomic nuclei to further processing, such as spatial encoding.

The anatomical significance of a functional network of the brain has an extremely important reference effect in a neurosurgery operation. For a patient, not only the cortical structures, but also the locations of the functional network always change due to various pathological changes. In order to locate the brain functional network, often an invasive cortical stimulation is made on an awake patient during the operation, but this method takes a long time, although it is widely used.

Functional magnetic resonance imaging (fMRI) is another modality often used to determine the functional network of a brain before the operation. Its basic mode is to perform image data acquisition when the patient is executing a specific task, such as language, memory or movement functions, followed by analyzing the acquired image before the operation in order to recognize the functionally active cortical area of the brain functional network. In recent years, it is also proposed to use the low frequency fluctuations of blood oxygen saturation level (BOLD) signals, measured in resting state fMRI, as a possible way of locating a number of functional networks simultaneously. However, the repeatability of the fMRI functional locations is still a problem, and the location results are not always consistent with the discoveries of the invasive method.

### SUMMARY OF THE INVENTION

An object of the present invention is to provide a method for determining an intracortical working state of a brain functional network.

The method according to the invention has a first acquisition step of acquiring a number of first blood oxygen saturation level time point vectors of a number of first gray matter voxels of a first cortex of a brain functional network template, each of the first blood oxygen saturation level time point vectors respectively having blood oxygen saturation level signals of each of the first gray matter voxels at a number of continuous time points in a particular time period. The method further has a first clustering step of clustering the first blood oxygen saturation level time point vectors as a number of first gray matter voxel cooperative time point categories by taking the blood oxygen saturation level signals as objects. The first gray matter voxel cooperative time point categories are sets of a number of discrete time points of the first gray matter voxels respectively in the particular time period. The method further has a first determination step of determining the number of first gray matter voxel cooperative time point categories as the intracortical working state of the first cortex.

Preferably, the first clustering step includes clustering the blood oxygen saturation level time point vectors as a number of first gray matter voxel cooperative time point categories by taking the blood oxygen saturation level signals as objects through a K-means algorithm.

Preferably, the first clustering step further includes traversing clustering results of a number of alternative K values through a first general formula so as to determine an optimal K value, the number of gray matter voxel cooperative time point categories including n gray matter voxel cooperative time point category pairs, and the gray matter voxel cooperative time point category pairs including two gray matter voxel cooperative time point categories whose working states are associated. The first general formula is

$$PI = {\sum }_{i = 1}^{n} {R}_{i} {P}_{i}$$

wherein Rᵢ is an absolute value of a correlation coefficient between the ⁱth gray matter voxel cooperative time point category pair, and Pᵢ is a percentage of the ⁱth gray matter voxel cooperative time point category pair in the particular time period, and the Pᵢ value corresponding to the maximum K value is taken as the optimal K value.

Preferably, the alternative K values comprise natural numbers greater than or equal to 4 and less than or equal to the radication of the number of the continuous time points.

Preferably, if Rᵢ is less than a threshold, Rᵢ is set as 0.

Preferably, said particular time period is repetition time of a scanning sequence of the magnetic resonance imaging system.

The present invention further provides a method for determining an intercortical working state of a brain functional network, that includes the method for determining an intracortical working state of as described above. This method further includes a second acquisition step of acquiring a number of second blood oxygen saturation level time point vectors of a number of second gray matter voxels of a second cortex of the brain functional network template, each of the second blood oxygen saturation level time point vectors respectively having blood oxygen saturation level signals of each of the second gray matter voxels at a number of continuous time points in the particular time period. The method further includes a second clustering step of clustering the number of second gray matter voxels of the second cortex of the brain functional network template in the particular time period as a number of second gray matter voxel cooperative time point categories according to the intracortical working state of the first cortex. The method further includes a judgment step of judging whether the respective corresponding number of second gray matter voxel cooperative time point categories and the number of first gray matter voxel cooperative time point categories have a number of cooperative relations, and a second determination step of determining the number of cooperative relations as the intercortical working state between said first cortex and the second cortex.

Preferably, the judgment step includes judging whether the respective corresponding number of second gray matter voxel cooperative time point categories and the number of first gray matter voxel cooperative time point categories have a number of cooperative relations by using a significance test approach.

Preferably, the significance test approach is a t-test, a z-test or chi-square test.

It can be seen from above-mentioned solutions that the method for determining an intracortical working state and an intercortical working state by a magnetic resonance imaging system according to a particular embodiment of the present invention have the following advantages: based on the dynamic activity characteristics of the brain functional network during no task state, the topography of the brain functional network and the amplitude and frequency of cortical activities are provided; the method for determining an intercortical working state by a magnetic resonance imaging system according to a particular embodiment of the present invention is a method which is non-invasive and has no task, and this method shows a better performance as compared with other rest state functional magnetic resonance data analysis methods for evaluating a brain function; the method for determining an intercortical working state by a magnetic resonance imaging system according to a particular embodiment of the present invention not only can calculate the spatial area of the brain functional network, but also can directly show the interactions in one cortex or among different cortices or different sub-cortices, and these interactions often appear in a very short time during the rest state and are difficult to be monitored by previous methods; when using a method for analyzing rest state functional magnetic resonance data collected by multilayer parallel acquisition accelerated echo planar imaging (EPI) technology, relatively high time resolution will bring more changes and decrease relativity of data in a relatively long time period; furthermore, the method for determining an intercortical working state by a magnetic resonance imaging system according to a particular embodiment of the present invention can study the dynamic state of the brain functional network using the advantage of the BOLD signals that have a relatively high time resolution.

### BRIEF DESCRIPTION OF THE DRAWINGS

FIG. 1 is a step diagram of the method for determining an intracortical working state according to an embodiment of the present invention.

FIG. 2 is a step diagram of the data preprocessing method in the method for determining an intracortical working state according to an embodiment of the present invention.

FIG. 3 is a step diagram of the method for determining an intercortical working state according to an embodiment of the present invention.

### DESCRIPTION OF THE PREFERRED EMBODIMENTS

In the fMRI field, the time related blood oxygen saturation level (BOLD) signals at relatively low frequency (0.1 Hz-0.01 Hz) collected by fMRI during the rest state are used to study human brains. Now, the analysis of the brain functional network in the rest state on the data in a single scan is often based on the hypothesis that the activities of the brain functional network do not change over time: calculating the linear correlation coefficient throughout the whole scan, and using the linear correlation coefficient to characterize the connection strength in the observation area. The particular method comprises: based on the analysis of the region of interest (ROI) of a seed (using the time sequence of the ROI as the regression factor to query the region having a similar time behavior throughout the brain) and the independent component analysis which is a no model approach, recognizing the spatial region having activities cooperated with time. Other methods for characterizing the brain functional network in the rest state comprises partial correlation, coherence and partial coherence, phase relation, spatial clustering and graph theory. Although some studies tried to calculate relevant patterning in a short time using a sliding window, the dynamic change presented by these methods still has an extremely low time resolution, and if the duration time of the time state of the brain functional network is shorter than the time window (often more than 1 min) for the algorithm, then these patterns will concentrate in the most frequent state in this time window. In conclusion, only the most common time state during a relatively long time period (more than 1 min) can be seen from all the local anatomical patterns of the brain functional network calculated by the above-mentioned methods. Other time states which appear at a relatively low probability will not be observed, and on the other hand, the variance of the most common state is raised.

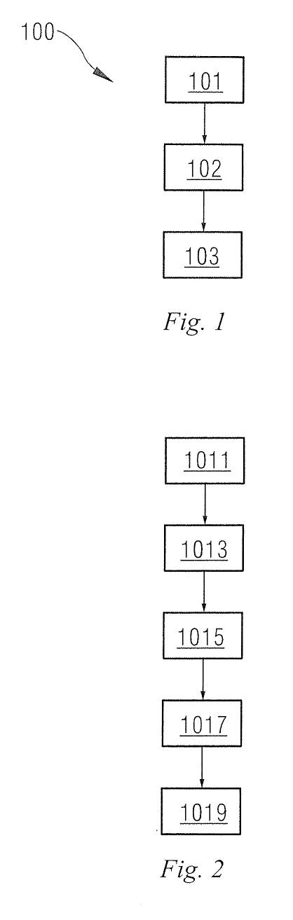

FIG. 1 is the step diagram of the method for determining an intracortical working state by a magnetic resonance imaging system according to a particular embodiment of the present invention. In view of the above-mentioned situation, as shown in FIG. 1, the present invention provides a method 100 for determining an intracortical working state of a brain functional network by a magnetic resonance imaging system, characterized in that the method comprises: a first acquisition step 101 of acquiring a number of first blood oxygen saturation level time point vectors of a number of first gray matter voxels of a first cortex of a brain functional network template, each of said first blood oxygen saturation level time point vectors respectively comprising blood oxygen saturation level signals of each of said first gray matter voxels at a number of continuous time points in a particular time period; a first clustering step 102 of clustering said first blood oxygen saturation level time point vectors as a number of first gray matter voxel cooperative time point categories, said first gray matter voxel cooperative time point categories being sets of a number of discrete time points of said first gray matter voxels having similar blood oxygen saturation level signals in said particular time period; and a first determination step 103 of determining said number of first gray matter voxel cooperative time point categories as said intracortical working state of said first cortex.

In particular, with a movement network mapping of a subject as an example, in the first acquisition step 101, a number of first blood oxygen saturation level time point vectors of a number of first gray matter voxels of a first cortex of a brain functional network template is acquired, each of said first blood oxygen saturation level time point vectors respectively comprising blood oxygen saturation level signals of each of said first gray matter voxels at a number of continuous time points in a particular time period.

FIG. 2 is the step diagram of the data preprocessing method in the method for determining an intracortical working state by a magnetic resonance imaging system according to a particular embodiment of the present invention. As shown in FIG. 2, in the method for determining an intracortical working state by a magnetic resonance imaging system according to a particular embodiment of the present invention, the standard data preprocessing method for analyzing the fMRI in the rest state will be applied to brain functional data collected during the no task time period of the subject, and the data preprocessing method comprises the following steps: step 1011, removing magnetic resonance imaging signals collected in the repetition time (TR) of the first 4 sequential scannings to guarantee the magnetic resonance imaging signals in a steady state; step 1013, performing image acquisition time correction and motion correction on the magnetic resonance imaging signals; step 1015, performing space smoothing on the magnetic resonance imaging signals (Gaussian kernel, 6 mm, maximum of the half of the full width) and eliminating the baseline drift of the magnetic resonance imaging signals; step 1017, eliminating the interferences of head movement, global signals, white matter signals and encephalocoele signals with the magnetic resonance imaging signals; and step 1019, extracting blood oxygen saturation level signals of a number of gray matter voxels at a number of continuous time points in a particular time period from the magnetic resonance imaging signals according to the sensory-motor cortex template of the brain functional network, thus constructing a number of blood oxygen saturation level time point vectors of a number of gray matter voxels of the sensory-motor cortex of a brain functional network template, where said blood oxygen saturation level time point vectors comprise blood oxygen saturation level signals of each of said gray matter voxels at a number of continuous time points in a particular time period.

The brain functional network template comprises a number of cortices, such as default mode, attention, vision, and sensory-motor networks, the method for determining an intracortical working state by a magnetic resonance imaging system according to a particular embodiment of the present invention uses the sensory-motor network as the example, and the present invention would also be able to be used for other cortices.

The brain functional network template can be obtained from rest state fMRI data of a number of general subjects, and can also be obtained from anatomical data of the brain functional network comprising precentral gyms, postcentral gyrus and central groove.

In particular, a repetition time TR of the scanning sequence of a magnetic resonance imaging system is used as said particular time period; therefore, said blood oxygen saturation level time point vectors comprise blood oxygen saturation level signals of each of said gray matter voxels at a number of continuous time points in the repetition time TR.

The first clustering step 102 consists in clustering said first blood oxygen saturation level time point vectors as a number of first gray matter voxel cooperative time point categories by taking the blood oxygen saturation level signals as objects, said first gray matter voxel cooperative time point categories being sets of a number of discrete time points of said first gray matter voxels in said particular time period.

Clustering analysis is an analysis process that groups sets of physical or abstract objects into a number of categories consisting of similar objects. In particular for the method for determining an intracortical working state by a magnetic resonance imaging system according to a particular embodiment of the present invention, the method for determining an intracortical working state by a magnetic resonance imaging system according to a particular embodiment of the present invention clusters various discrete time points of the respective first gray matter voxel by taking the blood oxygen saturation level signals as objects, that is to say, various discrete time points of the respective first gray matter voxel having a similar blood oxygen saturation level signal are combined into a number of categories in the method. The clustering analysis comprises a number of algorithms, such as K-mean value algorithm, K-MEDOIDS algorithm, CLARANS algorithm, BIRCH algorithm, CURE algorithm, and CHAMELEON algorithm.

The method for determining an intracortical working state by a magnetic resonance imaging system according to a particular embodiment of the present invention clusters said blood oxygen saturation level time point vectors as a number of first gray matter voxel cooperative time point categories by taking said blood oxygen saturation level signals as objects using a K-means algorithm. In the method, K is the number of first gray matter voxel cooperative time point categories.

Said first clustering step 102 further comprises traversing clustering results of a number of alternative K values through a first general formula so as to determine an optimal K value, said number of gray matter voxel cooperative time point categories comprises n gray matter voxel cooperative time point category pairs, and said gray matter voxel cooperative time point category pairs comprise two gray matter voxel cooperative time point categories whose working states are associated, and said first general formula is

$$PI = {\sum }_{i = 1}^{n} {R}_{i} {P}_{i}$$

in the formula, Rᵢ is an absolute value of a correlation coefficient between the ⁱth gray matter voxel cooperative time point category pair, and Pᵢ is a percentage of the ⁱth gray matter voxel cooperative time point category pair in said particular time period, and the K value corresponding to the maximum PI value is taken as said optimal K value. In the method, a correlation coefficient between the ⁱth gray matter voxel cooperative time point category pair (such as Pearson correlation coefficient, for detecting whether two data sets are on one line so as to measure the linear relation between interval variables or other correlation coefficient) is calculated, i.e., averaging/statistic tests are performed on the ⁱth gray matter voxel cooperative time point category pair according to the time point, thereby obtaining the standard state of the working state related to said gray matter voxel cooperative time point category, then the absolute value Rᵢ of the correlation coefficient is calculated.

In the method, said gray matter voxel cooperative time point category pair comprises the gray matter voxel cooperative time point category related to an activated working state and the gray matter voxel cooperative time point category related to an inhibited working state. When the paired working states have a high correlation coefficient, and the ratio of all the time appearing successfully paired working states accounting for the total time is high, i.e. the pairing effect is good.

If Rᵢ is less than a threshold (such as 0.5), Rᵢ is set as 0. As such, the paired working states having a relative low correlation coefficient are omitted so as to improve the accuracy and efficiency.

Said alternative K values comprise natural numbers greater than or equal to 4 and less than or equal to the radication of the number of said continuous time points or said discrete time points, the K value corresponding to the maximum PI value is taken as the optimal K value.

The first determination step 103 consists in determining said number of first gray matter voxel cooperative time point categories as said intracortical working state of said first cortex.

The method for determining an intracortical working state by a magnetic resonance imaging system according to a particular embodiment of the present invention will determine said number of first gray matter voxel cooperative time point categories obtained by using the optimal K value as said intracortical working state of said first cortex.

In conclusion, the method for determining an intracortical working state by a magnetic resonance imaging system according to a particular embodiment of the present invention can obtain changes of the brain functional network over time, and further classify the specific cortices of the brain functional network by the working states.

The method for determining an intercortical working state by a magnetic resonance imaging system according to a particular embodiment of the present invention is based on the hypothesis that the brain functional network is dynamically working: BOLD signals highly relate to high frequency power envelopes observed by electrophysiological sound recording technology. It can be seen from behavior and electrophysiological studies that the interactions or dynamic changes of intercortical working states of the brain functional network are much faster than 5 min. However, the methods in existing fMRI studies can only show the most common time state during a relatively long time period (more than 1 min).

Limited by the time resolution of fMRI imaging, fMRI data in the repetition time TR often only have 100-200 time points. Although there are controversies on the cortex number of the brain functional network and how to define cortices, the brain functional network has at least 5 to 7 cortices, such as default mode, attention, vision, and sensory-motor cortices, and each cortex has a number of time states (at least two states: activated and inhibited). Therefore, due to too small a number of time points, the interaction states among the cortices of the brain functional network are difficult to achieve a static state. In view of this, the method for determining an intercortical working state by a magnetic resonance imaging system according to a particular embodiment of the present invention uses the method for determining an intracortical working state by a magnetic resonance imaging system according to a particular embodiment of the present invention, i.e. firstly focusing on one of the cortices of the brain functional network, thereby reducing the dimension so as to perform K-means clustering, then generalizing the clustered results to other cortices, thus performing a whole brain statistical analysis of the clusters of the respective working state among the cortices having the identical working state.

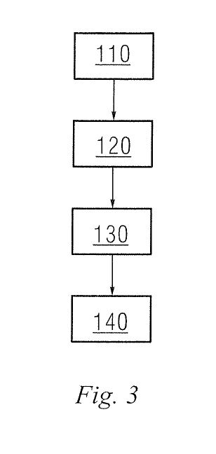

FIG. 3 is the step diagram of the method for determining an intercortical working state by a magnetic resonance imaging system according to a particular embodiment of the present invention. As shown in FIG. 3, the method for determining an intercortical working state by a magnetic resonance imaging system according to a particular embodiment of the present invention comprises: the method 100 for determining an intracortical working state as mentioned above; a second acquisition step 110 of acquiring a number of second blood oxygen saturation level time point vectors of a number of second gray matter voxels of a second cortex of said brain functional network template, each of said second blood oxygen saturation level time point vectors respectively comprising blood oxygen saturation level signals of each of said second gray matter voxels at a number of continuous time points in said particular time period; a second clustering step 120 of clustering said second blood oxygen saturation level time point vectors as a number of second gray matter voxel cooperative time point categories according to said intracortical working state; a judgment step 130 of judging whether the respective corresponding number of second gray matter voxel cooperative time point categories and said number of first gray matter voxel cooperative time point categories have a number of cooperative relations; and a second determination step 140 of determining said number of cooperative relations as said intercortical working state between said first cortex and said second cortex.

In particular, the working states between the sensory-motor cortex and visual cortex are used as the examples. Firstly the intracortical working state of the sensory-motor cortex is obtained according to the method for determining an intracortical working state by a magnetic resonance imaging system according to a particular embodiment of the present invention.

The second acquisition step 110 consists in acquiring a number of second blood oxygen saturation level time point vectors of a number of second gray matter voxels of a second cortex of said brain functional network template, each of said second blood oxygen saturation level time point vectors respectively comprising blood oxygen saturation level signals of each of said second gray matter voxels at a number of continuous time points in said particular time period.

The second clustering step 120 consists in clustering a number of second gray matter voxels of the visual cortex (the second cortex) in the particular time period as a number of second gray matter voxel cooperative time point categories according to the intracortical working state of the sensory-motor cortex (the first cortex); that is, clustering the second blood oxygen saturation level time point vectors as a number of second gray matter voxel cooperative time point categories according to sets of a number of discrete time points of said number of first gray matter voxel cooperative time point categories.

The judgment step 130 consists in judging whether the respective corresponding number of second gray matter voxel cooperative time point categories and said number of first gray matter voxel cooperative time point categories have a number of cooperative relations.

In the method, said judgment step comprises judging whether the respective corresponding number of second gray matter voxel cooperative time point categories and said number of first gray matter voxel cooperative time point categories have a number of cooperative relations by using a significance test approach. In the method, said significance test approach comprises t test, z test or chi-square test. In the method, the cooperative relation means that different cortices have an identical or similar working state at an identical time point, and this can be judged according to the method for determining an intercortical working state by a magnetic resonance imaging system according to a particular embodiment of the present invention, based on whether said second gray matter voxel cooperative time point categories are identical or similar to said first gray matter voxel cooperative time point categories.

Significance test is that firstly a hypothesis is made on the overall (random variables) parameters or general distribution form, followed by judging whether this hypothesis (alternative hypothesis) is reasonable using the sample information, i.e, judging whether there is a significant difference between the overall actual situation and the original hypothesis. For the method for determining an intercortical working state by a magnetic resonance imaging system according to a particular embodiment of the present invention, the test is that it is assumed that the respective corresponding number of second gray matter voxel cooperative time point categories and said number of first gray matter voxel cooperative time point categories have a number of cooperative relations, then said cooperative relations are judged by the significance test.

The method for determining an intercortical working state by a magnetic resonance imaging system according to a particular embodiment of the present invention uses a t test approach, and for repetition time (TR) of each working state, the t statistical value of each gray matter voxel is calculated by traversing all continuous time points or discrete time points in the repetition time to form the working state of the whole brain.

The second determination step 140 consists in determining said number of cooperative relations as said intercortical working state between said first cortex and said second cortex.

During the rest state scanning, there are a few constraint conditions on the cognitive process of the subject, and therefore it is possible that the dynamic change of respective cortex of the brain functional network can reflect the changes or awaking of the brain. It is also possible that the obvious changes are only driven by noises (such as movement and physiology) at a given time. In the present invention, t test corresponding to the time point category of respective working state cluster (of a time point) applied to respective gray matter voxel of the whole brain can screen noise states and show the gray matter voxel having the identical respective working state (of a time point). Intercortical working state not only shows different working states in a cortex, but also shows the stable interactions with other cortices so as to execute a cognitive task.

The method for determining an intracortical working state and an intercortical working state by a magnetic resonance imaging system according to a particular embodiment of the present invention has the following advantages: based on the dynamic activity characteristics of the brain functional network during no task state, the topography of the brain functional network and the amplitude and frequency of cortical activities are provided; the method for determining an intercortical working state by a magnetic resonance imaging system according to a particular embodiment of the present invention is a method which is non-invasive and has no task, and this method shows a better performance as compared with other rest state functional magnetic resonance data analysis methods for evaluating a brain function; the method for determining an intercortical working state by a magnetic resonance imaging system according to a particular embodiment of the present invention not only can calculate the spatial area of the brain functional network, but also can directly show the interactions in one cortex or among different cortices or different sub-cortices, and these interactions often appear in a very short time during the rest state and are difficult to be monitored by previous methods; when using a method for analyzing rest state functional magnetic resonance data collected by multilayer parallel acquisition accelerated echo planar imaging (EPI) technology, relatively high time resolution will bring more changes and decrease relativity of data in a relative long time period; furthermore, the method for determining an intercortical working state by a magnetic resonance imaging system according to a particular embodiment of the present invention can study the dynamic state of the brain functional network using the advantage of the BOLD signals that have a relatively high time resolution.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for determining an intracortical working state of a brain functional network, characterized in that the method comprises:
a first acquisition step of acquiring a plurality of first blood oxygen saturation level time point vectors of a plurality of first gray matter voxels of a first cortex of a brain functional network template, each of said first blood oxygen saturation level time point vectors respectively comprising blood oxygen saturation level signals of each of said first gray matter voxels at a plurality of continuous time points in a particular time period;
a first clustering step of clustering said first blood oxygen saturation level time point vectors as a plurality of first gray matter voxel cooperative time point categories by taking the blood oxygen saturation level signals as objects, said first gray matter voxel cooperative time point categories being sets of a plurality of discrete time points of said first gray matter voxels in said particular time period; and
a first determination step of determining said plurality of first gray matter voxel cooperative time point categories as said intracortical working state of said first cortex.

2. The method for determining an intracortical working state of claim 1, wherein said first clustering step comprises clustering said first blood oxygen saturation level time point vectors as a plurality of first gray matter voxel cooperative time point categories by taking the blood oxygen saturation level signals as objects through a K-means algorithm.

3. The method for determining an intracortical working state of claim 2, wherein said first clustering step further comprises traversing clustering results of a plurality of alternative K values through a first general formula so as to determine an optimal K value, the plurality of gray matter voxel cooperative time point categories comprises n gray matter voxel cooperative time point category pairs, said gray matter voxel cooperative time point category pairs comprise two gray matter voxel cooperative time point categories whose working states are associated, and the first general formula is
$$PI = {\sum }_{i = 1}^{n} {R}_{i} {P}_{i}$$
In the formula, Rᵢ is an absolute value of a correlation coefficient between the ⁱth gray matter voxel cooperative time point category pair, and Pᵢ is a percentage of the ⁱth gray matter voxel cooperative time point category pair in said particular time period, and the K value corresponding to the maximum PI value is taken as said optimal K value.

4. The method for determining an intracortical working state of claim 3, wherein said alternative K values comprise natural numbers greater than or equal to 4 and less than or equal to the radication of the number of said continuous time points.

5. The method for determining an intracortical working state of claim 3, wherein if Rᵢ is less than a threshold, then Rᵢ is set as 0.

6. The method for determining an intracortical working state of claim 1, wherein said particular time period is repetition time of a scanning sequence of a magnetic resonance imaging system.

7. A method for determining an intercortical working state of a brain functional network, comprising:
a second acquisition step of acquiring a plurality of second blood oxygen saturation level time point vectors of a plurality of second gray matter voxels of a second cortex of said brain functional network template, each of said second blood oxygen saturation level time point vectors respectively comprising blood oxygen saturation level signals of each of said second gray matter voxels at a plurality of continuous time points in said particular time period;
a second clustering step of clustering the plurality of second gray matter voxels of the second cortex of said brain functional network template in said particular time period as a plurality of second gray matter voxel cooperative time point categories according to said intracortical working state of said first cortex;
a judgment step of judging whether the respective corresponding plurality of second gray matter voxel cooperative time point categories and said plurality of first gray matter voxel cooperative time point categories have a plurality of cooperative relations; and
a second determination step of determining said plurality of cooperative relations as said intercortical working state between said first cortex and said second cortex.

8. The method for determining an intercortical working state of claim 7, wherein said judgment step comprises judging whether the respective corresponding plurality of second gray matter voxel cooperative time point categories and said plurality of first gray matter voxel cooperative time point categories have a plurality of cooperative relations by using a significance test approach.

9. The method for determining an intercortical working state of claim 8, wherein said significance test approach comprises t test, z test or chi-square test.
